(19) United States
(12) Patent Application Publication
(10) Pub. No.: US 2023/0218200 A1
(43) Pub. Date: Jul. 13, 2023

(54) SECONDARY VESTIBULAR SYSTEM FALL PREVENTATIVE DEVICE

(71) Applicant: Alexandra Miller-Davey, Bolton, CT (US)

(72) Inventor: Alexandra Miller-Davey, Bolton, CT (US)

(21) Appl. No.: 17/570,768

(22) Filed: Jan. 7, 2022

(51) Int. Cl. A61B 5/11; A61B 5/00

(52) U.S. Cl. CPC A61B 5/1117; A61B 5/6822; A61B 5/746; A61B 5/0053; A61B 2562/0219

(57) ABSTRACT

A wearable fall prevention device and method including at least one gyroscope, an actuator, a pressure applicator, and a controller. The at least one gyroscope is configured to detect and signal to a controller when a user's movement and/or orientation surpasses a specified threshold. The controller receives the signal from the at least one gyroscope and generates an actuation signal received by the at least one actuator, activating the at least one pressure applicator. The at least one pressure applicator is adapted to come into contact with the user's neck and vagus nerves at a specified pressure. When the at least one gyroscope detects a user has dropped below the specified threshold, the at least one gyroscope sends a signal to the controller, generating an actuation signal that deactivates the at least one actuator and disengages the at least one pressure applicator from the user's neck and vagus nerves.

### TECHNICAL FIELD

The present disclosure relates generally to wearable fall prevention systems, devices and methods.

### BACKGROUND

As an individual ages, blood vessel walls tend to thicken due to an aging process known as arteriosclerosis. This thickening results in partial occlusion, with a gradual decrease of blood flow to the inner ear structures. The balance mechanism of the vestibular system usually adjusts to this, but at times persistent unsteadiness develops. Unsteadiness can cause a user to fall, resulting in minor to serious injuries.

### SUMMARY

According to one embodiment of the present disclosure, a fall preventative device worn by a user comprises at least one gyroscope, wherein the gyroscope senses the movement and/or orientation of the user about two distinct axes, at least one actuator, at least one spring, at least one output shaft located within the actuator, at least one pressure applicator. The gyroscope is configured to detect and signal when a user's movement and/or orientation exceeds a certain orientation threshold. The controller is configured to receive a movement and/or orientation signal from the gyroscope and generates an actuation signal, activating the actuator and causing the output shaft to release the spring and thrust the pressure applicator into contact with a user's neck and the vagus nerves at a specified pressure. The pressure provides an early stimulation of the vagus nerves and enables to user to detect an imminent fall and react to prevent a fall.

According to the present disclosure, a wearable fall preventative device comprising at least one gyroscope, wherein the gyroscope senses the movement and/or orientation of the user about two distinct axes, at least one actuator, at least one spring, an output shaft located within the actuator and at least one pressure applicator. The gyroscope is configured to generate and signal when a user's movement and/or orientation drops below a specified orientation threshold. The controller receives the signal from the gyroscope and generates actuation signals, deactivating the actuator and disengaging the output shaft and pressure applicator from the user's neck and vagus nerves.

According to the present disclosure, a preventative fall method includes the steps of monitoring, by at least one gyroscope, the movement and/or orientation of a user, determining, by the gyroscope, if the movement and/or orientation of a user exceeds a specified orientation threshold, and generating, by the controller, a signal to initiate the actuator, the output shaft, the spring, and the pressure applicator, wherein the pressure applicator comes into contact with the user's neck and vagus nerves at a specified pressure.

These and other aspects, features and advantages of the present disclosure will become apparent in light of the following detailed description of non-limiting embodiments, with reference to the accompanying drawings.

### BRIEF DESCRIPTION OF THE DRAWINGS

FIG. 1 is a schematic view of an exemplary wearable fall prevention device in accordance with one embodiment of the present disclosure;

FIG. 2 is a top view of the actuators of the device of FIG. 1 in the retracted position in the absence of an actuating signal in accordance with the present disclosure;

FIG. 3 is a top view of the actuators of FIG. 2 in the extended position applying pressure to the vagus nerves of the user after receiving an actuating signal in accordance with the present disclosure;

FIG. 4 is a flow chart illustrating the operation of a controller configured to be executed by the device of FIG. 1 of the present disclosure; and

FIG. 5 is a flow chart illustrating an exemplary fall prevention algorithm configured to be executed by the wearable fall prevention device of FIG. 1 in accordance with embodiments of the present disclosure.

### DETAILED DESCRIPTION

Before the various embodiments are described in further detail, it is to be understood that the present disclosure is not limited to the particular embodiments described. It will be understood by one of ordinary skill in the art that the systems and methods described herein may be adapted and modified as is appropriate for the application being addressed and that the systems and methods described herein may be employed in other suitable applications, and that such other additions and modifications will not depart from the scope thereof.

Although various features have been shown in different figures for simplicity, it should be readily apparent to one of skill in the art that the various features may be combined without departing from the scope of the present disclosure.

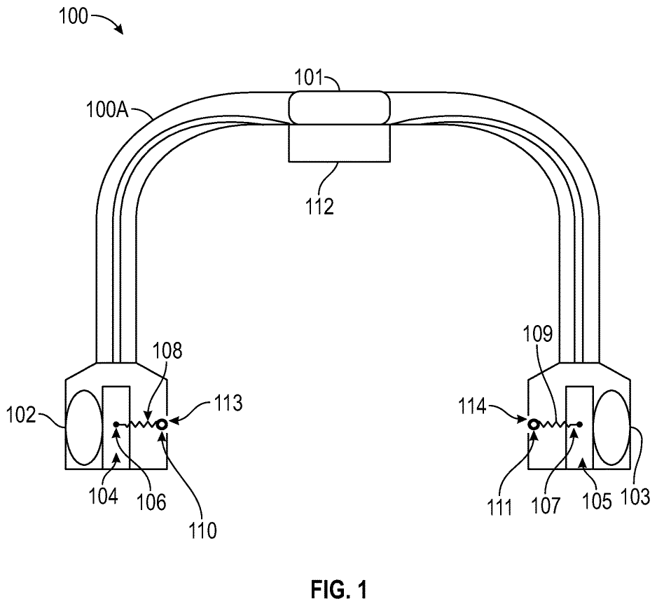

Referring to FIG. 1, exemplary wearable fall preventative device 100 including a device body 100A having at least one gyroscope (in this preferred embodiment, three gyroscopes 101, 102, 103), at least one actuator mechanism (in this embodiment first and second actuators 104, 105), and at least one pressure applicator (in this embodiment two pressure applicators being formed by two output shafts 106, 107, two springs 108, 109, two pressure applicators 110, 111), a controller 112, and two recesses 113, 114. Other embodiments can have one actuator on either the left side or right side of the user's neck and vice-versa. The device body is elongated and has one end and an opposite end. The first of the gyroscopes 101 is positioned in the middle of the device, straddling the user's neck, flush against the outer wall of the device, the second gyroscope 102 is positioned on one end of the device, straddling the user's neck, flush against the outer wall of the device, and the third gyroscope 103 is placed on the opposite end of the device, straddling the user's neck, flush against the outer wall of the device. The first actuator 104 is positioned in the middle of one end of the device, with one side connected to the controller 112 and the other side flush against the end wall of the device. The second actuator 105 is positioned in the middle of the other end of the device, with one side connected to the controller 112 and the other side flush against the end wall of the device. The output shafts 106 and 107 are attached to actuators 104 and 105 as well as attached to springs 108 and 109. The pressure applicators 110 and 111 are connected to springs 108, 109, and are positioned flush against the inner wall recesses 113, 114 of the device. The controller 112 is configured to receive signals and/or data from the gyroscopes 101, 102, 103 and generate/send signals to actuators 104, 105. As shown, each gyroscope 101, 102, 103 are positioned in the middle, one end, and the opposite end of the device 100. However, the gyroscopes 101, 102, 103 may also be positioned in other areas of the device 100, for example, in intermediate areas (not shown) from where the gyroscopes 101, 102, 103 are currently positioned. Alternatively, there could be more than three gyroscopes positioned throughout the device 100, or less than three gyroscopes positioned throughout the device 100.

The gyroscopes 101, 102, 103 are configured to detect and signal when a user's movement and/or orientation exceeds a specified orientation threshold. The controller 112 is configured to receive movement and/or orientation signals from gyroscopes 101, 102, 103 and generate actuation signals to activate actuators 104 and 105, engage output shafts 106, 107, release springs 108, 109 and thrust pressure applicator(s) 110, 111 from recesses 113 and 114, thereby coming into contact with a user's neck and vagus nerves at a specified pressure. The actuators 104, 105 can receive signals from the controller 112 through wired connections or, alternatively, through wireless connections. FIG. 1 shows markings for a wired connection between gyroscopes 101, 102, and 103, actuators 104, 105, and controller 112.

The gyroscopes 101, 102, and 103 are also configured to detect and signal when a user's movement and/or orientation drops below a specified orientation threshold. The controller 112 is configured to receive the signals from gyroscopes 101, 102, 103 and generate actuation signals to deactivate actuators 104 and 105, disengage output shafts 106, 107, coil springs 108, 109 and allowing pressure applicator(s) 110, 111 to disengage the user's neck and vagus nerves and return back into recesses 113 and 114 to their default positions. The actuators 104, 105 can receive signals from gyroscopes 101, 102, 103 through wired connections or, alternatively, through wireless connections. FIG. 1 shows markings for a wired connection between gyroscopes 101, 102, 103, actuators 104, 105, and controller 112.

The gyroscopes 101, 102, 103 may utilize any type of gyroscope technology. For instance, the gyroscopes may be ring laser, fiber-optic, quantum, or vibration gyroscopes 101, 102, 103. The gyroscopes are configured to generate movement and/or orientation signals, which are capable of being detected or received by the controller 112, when the user's movement and/or orientation exceeds a certain threshold. The threshold can be predetermined and/or adjusted based on the user's age, mobility, and previous fall history.

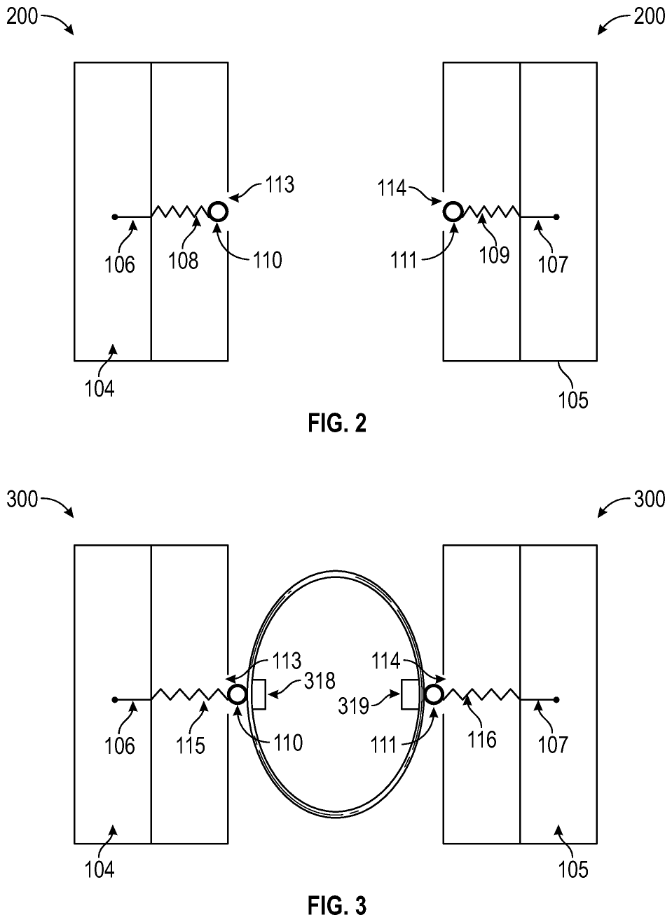

FIG. 2 shows an enlarged view 200 of the actuators 104, 105 before engagement with a user's neck and vagus nerves. The actuators 104, 105 have yet to receive a signal from the controller 112, and, thus, the output shafts 106, 107 have not engaged with coils 108, 109 and the pressure applicator(s) 110, 111 remain retracted inside recesses 113, 114.

FIG. 3 shows an enlarged view 300 of the servo motors 104, 105 after engagement with a user's neck. The actuators 104, 105 have received a signal from the controller 112, and, thus, the output shafts 106, 107 have engaged with coils 108, 109, which in turn thrusts pressure applicator(s) 110, 111 out of recesses 113, 114 and comes into contact with a user's neck 317 at vagus nerves 318, 319 of the user at a specified pressure.

In operation, the controller 112 is configured to determine if a fall event is imminent. An imminent fall event occurs when a user's movement and/or orientation exceeds a specified orientation threshold that would indicate that a fall event is occurring, likely to occur or imminent. This will trigger a response from the controller 112 as outlined above.

The present disclosure advantageously provides a device 100 capable of storing various acceleration and/or orientation values through calibration or manual selection. This is advantageous because not every user will have the same orientation value threshold that indicates a fall event is imminent. Thus, the controller 112 may be configured to disregard certain orientation values below a preselected value to avoid triggering a false preventative fall determination.

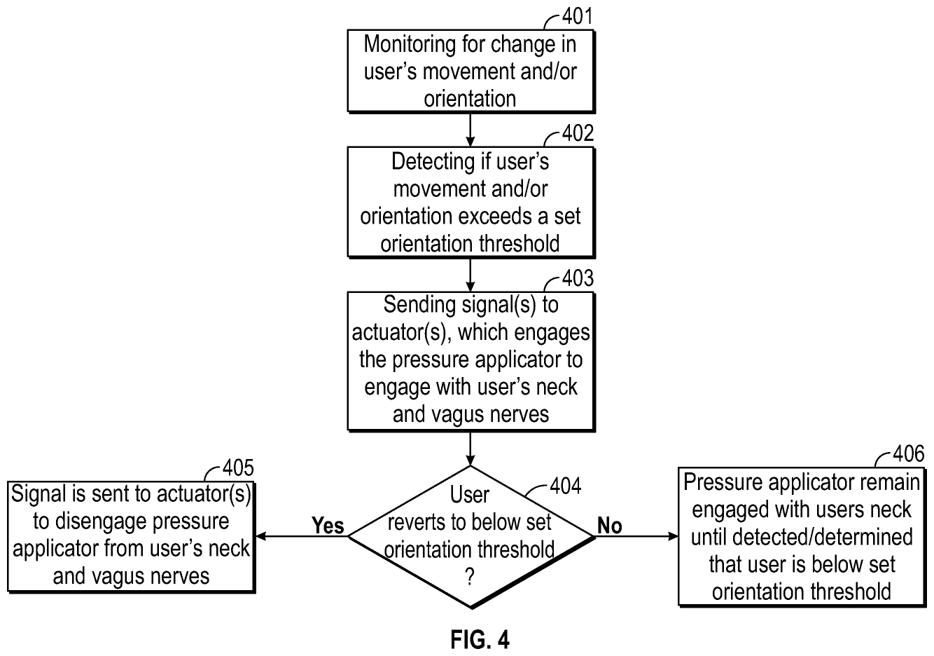

Referring to FIG. 4, the wearable fall prevention device 100 may be adapted to execute the steps illustrated in exemplary method 400 in order to monitor the user's movement and/or orientation and engage the fall prevention device 100 for a user if necessary. The exemplary method 400 includes the steps of monitoring 401 by the controller 112 for changes in the user's movement and/or orientation; detecting 402 with gyroscopes 101, 102, and 103 that a user's orientation exceeds a specified orientation threshold; sending 403 a signal from the controller 112 to actuator mechanism 200, which activates actuator mechanism 300 and engages pressure applicators 110 and 111 to come into contact with user's neck 117 at vagus nerves 318, 319 of the user at a specified pressure. If gyroscopes 101, 102 and 103 detect that the user's movement and/or orientation has dropped below a specified orientation threshold 404, gyroscopes 101, 102 and 103 will send a signal to controller 112, whereby a signal 405 is generated and sent from the controller 112 to deactivate the actuators 104, 105 and disengage pressure applicators 110 and 111 from user's neck 117 and vagus nerves 318, 319 and return to the default positions. If the user has not returned to below specific orientation threshold 406, then the pressure applicator(s) 110, 111 remain engaged with users neck 117 at vagus nerves 318, 319 (FIG. 3) of the user until the gyroscopes 101, 102 and 103 detect and signal to controller 112 that a user has fallen below a specified orientation threshold.

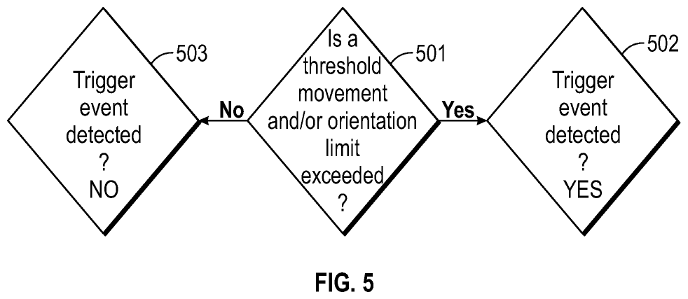

An exemplary algorithm executable by the controller 112 for detecting if a fall is imminent thus detecting a trigger event 502 or not detecting a trigger event 504 is illustrated in FIG. 5. If a threshold movement and/or orientation limit is exceeded 500, then a trigger event will be detected 502 and enable a user to detect and prevent an imminent fall by stimulating the vagus nerves. If a threshold orientation limit is not exceeded 500, then a trigger event will not occur 504.

In operation, the wearable fall prevention device 100 requires at least one gyroscope. More gyroscopes incorporated into the wearable fall prevention device 100 will allow for more movement signals capable of being detected. Thus, more orientation and movement data may be provided by the gyroscopes during operation, which may provide better operation of the wearable fall prevention device 100 by decreasing probability of a false positive determination of an imminent fall and/or false negative determinations of an imminent fall.

The present disclosure advantageously provides a wearable fall prevention device 100 and method 400 that does not require affirmative action by the user in order to receive an early warming of an imminent fall. This feature is advantageous at least because in some instances a user may not have time to recognize an imminent fall and take preventative action.

The exemplary wearable fall prevention device 100 and method 400 is shown and described as including three gyroscopes 101, 102, 103, two actuators 104, 105, two output shafts 106, 107, two springs 108, 109, two pressure applicators 110, 111, a controller 112, two recesses 113, 114, however, it should be understood that a wearable fall prevention device 100 and method 400 in accordance with the principles of the present disclosure can be designed with any number of gyroscopes, servo motors, output shafts, springs, pressure applicators, controllers, and recesses.

Advantageously, the device and method according to the present application serves as a secondary vestibular system to users whose primary vestibular system is aging or otherwise impaired. The secondary vestibular device and method in accordance with the present disclosure are configured to stimulate the vagus nerves of a user (e.g. with applied pressure) to cause a user response that would otherwise not occur (or not occur in a timely fashion) because of, for example, arteriosclerosis.

While the present disclosure has been illustrated and described with respect to particular embodiments thereof, it should be appreciated by those of ordinary skill in the art that various modifications of this disclosure may be made without departing from the spirit and scope of the present disclosure. For example, while the present disclosure provides a wearable fall prevention device system well-suited for elderly user use, it should be readily understood that principles of the present disclosure can be applied to applications where, for example, users are not elderly.

What is claimed is:

1. A wearable fall preventative device comprising:
a device body configured to be worn by a user;
at least one gyroscope connected to the device body and configured to detect movement and/or orientation of the user;
at least one pressure applicator connected to the device body;
at least one actuator connected in controlling relationship with the pressure applicator; and
a controller configured to receive movement data and/or orientation data from the at least one gyroscope, and configured to control the pressure applicator by way of the actuator.

2. The wearable fall preventative device according to claim 1, wherein the device body is configured to be worn on a neck of the user.

3. The wearable fall preventative device according to claim 1, wherein the at least one pressure applicator is configured to apply a pressure to a neck of the user.

4. The wearable fall preventative device of claim 1, wherein the device body is elongated with one end and an opposite end, and the at least one gyroscope is arranged at a midpoint between the ends of the device body.

5. The wearable fall preventative device of claim 1, wherein the device body is elongated and has one end and an opposite end, and the at least one gyroscope includes a first gyroscope, positioned on the one end of the device body.

6. The wearable fall preventative device of claim 5, wherein the at least one gyroscope further includes a second gyroscope, positioned on the opposite end of the device body.

7. The wearable fall preventative device of claim 1, wherein the device body is elongated and has one end and an opposite end, and the at least one actuator includes a first actuator, positioned on the one end of the device body.

8. The wearable fall preventative device of claim 7, wherein the at least one actuator further includes a second actuator, and the second actuator is located on the opposite end of the device body.

9. The wearable fall preventative device of claim 1, wherein the at least one gyroscope includes a first, a second, and a third gyroscope, all of which are capable of storing various acceleration and orientation values.

10. The wearable fall preventative device of claim 1, wherein the at least one gyroscope includes a first, second, and a third gyroscope, wherein the first, second, and third gyroscopes are configured to detect when a user's motion surpasses a specified movement and/or orientation threshold.

11. The wearable fall preventative device of claim 1, wherein the at least one gyroscope includes a first, a second, and a third gyroscope, the first, second, and third gyroscopes are configured to detect and signal when a user's movement and/or orientation surpasses a specified threshold, the controller is in communication with the first, second, and third gyroscopes, and is configured to receive movement and/or orientation signals from the first, second and third gyroscopes to generate actuation signals, the at least one actuator includes a first and a second actuator receiving the actuation signals from the controller to activate the first and second actuators, and the at least one pressure applicator includes a first and a second pressure applicator responsive respectively to the first and second actuators to come into contact with the user's neck and vagus nerves at a specified pressure.

12. The wearable fall preventative device of claim 11, wherein the first, second, and third gyroscopes are configured to detect and signal when a user's movement and/or orientation drops below a specified orientation threshold, and in response the controller deactivates the first and second actuators, and the first and second pressure applicators disengage from the user's neck and vagus nerves.

13. The wearable fall preventative device of claim 1, wherein the at least one gyroscope includes a first, second, and a third gyroscope distributed on the device body, and a plurality of additional force sensors are positioned on the device body between the existing first, second, and third gyroscopes and generate additional movement and/or orientation signals, and the controller receives and is responsive to the additional orientation signals.

14. The wearable fall preventative device of claim 1, wherein the at least one gyroscope includes a first, a second, and a third gyroscope, the first, second, and third gyroscopes are configured to detect movements and/or orientations of different magnitudes and said controller determines if the orientation magnitude is above a specified orientation threshold which indicates the user is about to have an imminent fall event, before causing the pressure applicator to apply pressure to the user.

15. The wearable fall prevention device of claim 1, wherein the at least one pressure applicator includes a first and a second applicator comprised of metal balls and springs.

16. A preventative fall method comprising the steps of:
monitoring, by gyroscopes worn by a user, changes in the user's orientation;
detecting by the gyroscopes if the user's movement and/or orientation exceeds a set orientation threshold;
and generating and sending a warning signal from the gyroscopes to a controller which causes an pressure applicator to come into contact with the user's neck and vagus nerves at a specified pressure.
